(19) United States
(12) Patent Application Publication
(10) Pub. No.: US 2026/0183535 A1
(43) Pub. Date: Jul. 2, 2026

(54) Electrode Assemblies for Applying Tumor Treating Fields (TTFields) to a Subject's Body, with Features that Prevent the Electrode Assemblies from Peeling Away from the Subject's Skin

(71) Applicant: Novocure GmbH, Baar (CH)

(72) Inventors: Noa HALAVEE, Haifa (IL); Elle Yaacobi, Haifa (IL); Dmitry Golom, Haifa (IL)

(73) Assignee: Novocure GmbH, Baar (CH)

(21) Appl. No.: 19/436,019

(22) Filed: Dec. 30, 2025

Related U.S. Application Data
(60) Provisional application No. 63/740,915, filed on Dec. 31, 2024.

(51) Int. Cl. A61N 1/04

(52) U.S. Cl. CPC A61N 1/0408; A61N 1/0476; A61N 1/0496

(57) ABSTRACT

Electrode assemblies for applying Tumor Treating Fields (TTFields) to a subject's body can employ a sheet of graphite to spread out the heat and electrical current over the surface of the electrode assembly. But because graphite does not stretch, normal movements of the subject's body in certain anatomical locations can cause the electrode assemblies to peel away from the subject's body. The electrode assemblies described herein are shaped and configured in ways that reduce this tendency to peel away from the subject's body. This can be accomplished by configuring the sheet of graphite so that it has a main section and two protruding sections that extend from the main section into adjacent quadrants of a Cartesian coordinate system, with a gap disposed between the two protruding sections.

### CROSS REFERENCE TO RELATED APPLICATIONS

This Application claims the benefit of U.S. Provisional Application 63/740,915, filed Dec. 31, 2024, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 kHz. The alternating electric fields are induced by electrode assemblies (also called transducer arrays) positioned on the subject's skin on opposite sides of the subject's body. When an AC voltage is applied between opposing electrode assemblies, an AC current is coupled through the electrode assemblies and into the subject's body, which induces the TTFields in a target region within the subject's body.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier (BBB) so that, e.g., chemotherapy drugs can reach the brain.

U.S. Pat. No. 12,114,991 describes some different prior art electrode assemblies that can be used to apply alternating electric fields to a subject's body. In one example, each electrode assembly includes a set of electrode elements, each of which includes (a) a metal layer and (b) a ceramic layer with a very high dielectric constant positioned between the metal layer and the subject's skin. In another example, each electrode assembly includes a flex circuit that includes a plurality of conductive pads on the front side of the flex circuit, and these conductive pads serve as electrode elements.

US Pub. No. 2023/0043071 describes positioning a sheet of graphite in front of the electrode elements in an electrode assembly to spread both heat and electrical current out over a larger area. And a layer of conductive adhesive or conductive hydrogel is positioned between the sheet of graphite and the subject's body, which holds (or helps to hold) the electrode assembly against the subject's skin.

### SUMMARY OF THE INVENTION

One aspect of the invention is directed to a first electrode assembly that comprises a sheet of graphite and a flexible PCB. The sheet of graphite includes a main section, a first protruding section, and a second protruding section, with a gap disposed between the first and second protruding sections. The first and second protruding sections each extend into adjacent quadrants of a Cartesian coordinate system whose origin is positioned in the main section and whose Y axis is aligned with a centerline of the gap. The sheet of graphite has a front face and a rear face. The main section includes a first region and a second region disposed on opposite sides of the Y axis, and the first and second protruding sections extend from the first and second regions, respectively. The first protruding section is at least one-quarter as long as a longest dimension of the first region in the Y direction, and the second protruding section is at least one-quarter as long as a longest dimension of the second region in the Y direction. The flexible PCB is positioned behind the rear face of the sheet of graphite. And the PCB has a front face facing toward the sheet of graphite, a first metal pad that is disposed on the front face of the PCB and positioned behind the first region, a second metal pad that is disposed on the front face of the PCB and positioned behind the second region, a third metal pad that is disposed on the front face of the PCB and positioned behind the first protruding section, and a fourth metal pad that is disposed on the front face of the PCB and positioned behind the second protruding section. The first and second metal pads of the PCB are affixed to the first and second regions of the sheet of graphite, respectively, by first and second regions of a conductive adhesive or conductive hydrogel. And the third and fourth metal pads of the PCB are affixed to the first and second protruding sections of the sheet of graphite, respectively, by third and fourth regions of a conductive adhesive or conductive hydrogel.

In some embodiments of the first electrode assembly, the PCB has a plurality of metal traces configured to form electrically conductive paths between the first metal pad, the second metal pad, the third metal pad, and the fourth metal pad. And these embodiments further comprise a conductive wire that is electrically connected to at least one of the first, second, third, and fourth metal pads.

In some embodiments of the first electrode assembly, the PCB has a plurality of metal traces configured to form electrically conductive paths between the first metal pad, the second metal pad, the third metal pad, and the fourth metal pad. And these embodiments further comprise a conductive wire that is electrically connected to at least one of the first, second, third, and fourth metal pads; and a layer of conductive adhesive or conductive hydrogel disposed on the front face of the sheet of graphite. Optionally, these embodiments may also further comprise a flexible backing positioned behind the PCB, wherein the flexible backing is configured to support the PCB and the sheet of graphite.

In some embodiments of the first electrode assembly, the PCB has a plurality of metal traces configured to form electrically conductive paths between the first metal pad, the second metal pad, the third metal pad, and the fourth metal pad. And these embodiments further comprise a conductive wire that is electrically connected to at least one of the first, second, third, and fourth metal pads; and a layer of conductive adhesive disposed on the front face of the sheet of graphite. Optionally, these embodiments may also further comprise a flexible backing positioned behind the PCB, wherein the flexible backing is configured to support the PCB and the sheet of graphite.

In some embodiments of the first electrode assembly, the sheet of graphite has at least one slit disposed therein, and the at least one slit is positioned to increase flexibility of the electrode assembly when the electrode assembly is adhered to a subject's body. Optionally, these embodiments can further comprise a layer of flexible foam material shaped, dimensioned, and positioned to cover a front side of all edges of the sheet of graphite, and all of the slits in the sheet of graphite.

In some embodiments of the first electrode assembly, the sheet of graphite has a central slit that extends inward from an outer edge of the main section that is opposite to the gap, wherein the central slit is aligned with the Y axis. Optionally, the central slit extends at least one-tenth way through the main section. Optionally, the embodiments described in this paragraph can further comprise a layer of flexible foam material shaped, dimensioned, and positioned to cover a front side of all edges of the sheet of graphite, and all of the slits in the sheet of graphite.

In some embodiments of the first electrode assembly, the first and second protruding sections are symmetric about the Y axis. Optionally, in these embodiments, the sheet of graphite has (i) a first slit positioned between the first region and the first protruding section and (ii) a second slit positioned between the second region and the second protruding section. The first and second slits extend inward from an outer edge of the sheet of graphite in directions that are perpendicular ±20° to the Y axis, and the first and second slits each extend at least one-tenth way through the main section.

In some embodiments of the first electrode assembly, the first and second protruding sections are symmetric about the Y axis. The sheet of graphite has (i) a first slit positioned between the first region and the first protruding section (ii) a second slit positioned between the second region and the second protruding section, and (iii) a central slit that extends inward from an outer edge of the main section that is opposite to the gap. The first and second slits extend inward from an outer edge of the sheet of graphite in directions that are perpendicular ±20° to the Y axis, and the first and second slits each extend at least one-tenth way through the main section. The central slit is aligned with the Y axis, and the central slit extends at least one-tenth way through the main section.

Optionally, in the embodiments described in the previous paragraph, the sheet of graphite has a first additional slit that extends in a proximal direction from a distal end of the first protruding section, and a second additional slit that extends in a proximal direction from a distal end of the second protruding section. Optionally, these embodiments can further comprise a layer of flexible foam material shaped, dimensioned, and positioned to cover a front side of all edges of the sheet of graphite, and all of the slits in the sheet of graphite.

Optionally, the embodiments described in the previous paragraph can further comprise a fifth metal pad that is disposed on the front face of the PCB and positioned behind the first protruding section, and a sixth metal pad that is disposed on the front face of the PCB and positioned behind the second protruding section, and optionally a layer of flexible foam material shaped, dimensioned, and positioned to cover a front side of all edges of the sheet of graphite, and all of the slits in the sheet of graphite.

In some embodiments of the first electrode assembly, the first protruding section is at least half as long as the longest dimension of the first region in the Y direction, and the second protruding section is at least half as long as the longest dimension of the second region in the Y direction. In some embodiments of the first electrode assembly, the first protruding section is at least as long as the longest dimension of the first region in the Y direction, and the second protruding section is at least as long as the longest dimension of the second region in the Y direction.

In some embodiments of the first electrode assembly, the main section, the first protruding section, and the second protruding section are collectively arranged in a configuration that is substantially U-shaped. In some embodiments of the first electrode assembly, the main section, the first protruding section, and the second protruding section are collectively arranged in a configuration that is substantially V-shaped or substantially rounded V-shaped. In some embodiments of the first electrode assembly, the sheet of graphite has an area between 75 and 125cm² . In some embodiments of the first electrode assembly, the sheet of graphite is a sheet of pyrolytic graphite, graphitized polymer, or graphite foil made from compressed high purity exfoliated mineral graphite.

In some embodiments of the first electrode assembly, the sheet of graphite has at least one slit or elongated cutout disposed therein, and the at least one slit or elongated cutout is positioned to increase flexibility of the electrode assembly when the electrode assembly is adhered to a subject's body.

In some embodiments of the first electrode assembly, the sheet of graphite has a central slit or elongated cutout that extends inward from an outer edge of the main section that is opposite to the gap, and the central slit or elongated cutout is aligned with the Y axis.

In some embodiments of the first electrode assembly, the sheet of graphite has (i) a first slit or elongated cutout positioned between the first region and the first protruding section and (ii) a second slit or elongated cutout positioned between the second region and the second protruding section. In these embodiments, the first and second slits or elongated cutouts extend inward from an outer edge of the sheet of graphite in directions that are perpendicular ±20° to the Y axis, and the first and second slits or elongated cutouts each extend at least one-tenth way through the main section.

Optionally, in the embodiments described in the previous paragraph, the sheet of graphite has a central slit or elongated cutout that extends inward from an outer edge of the main section that is opposite to the gap, the central slit or elongated cutout is aligned with the Y axis, and the central slit or elongated cutout extends at least one-tenth way through the main section.

Some embodiments of the first apparatus further comprise a flexible backing positioned behind the PCB and a layer of conductive adhesive. In these embodiments, the flexible backing is configured to support the PCB and the sheet of graphite, and the flexible backing extends beyond an outer perimeter of the sheet of graphite. The layer of conductive adhesive is disposed on the front face of the sheet of graphite, and the layer of conductive adhesive covers all front edges of the sheet of graphite. And at least a first region of the flexible backing that extends beyond the outer perimeter of the sheet of graphite has a self-adhesive front face that is configured to adhere to skin.

Optionally, in the embodiments described in the previous paragraph, the sheet of graphite has at least one elongated cutout disposed therein, with the at least one elongated cutout being positioned to increase flexibility of the electrode assembly when the electrode assembly is adhered to a subject's body. At least a second region of the flexible backing that is disposed behind the at least one elongated cutout has a self-adhesive front face that is configured to adhere to skin. And the at least one elongated cutout is wide enough to permit the second region of the flexible backing to contact the subject's skin.

Some embodiments of the first apparatus further comprise a flexible backing positioned behind the PCB and a layer of conductive adhesive. In these embodiments, the flexible backing is configured to support the PCB and the sheet of graphite, and the flexible backing extends beyond an outer perimeter of the sheet of graphite. The layer of conductive adhesive is disposed on the front face of the sheet of graphite, and the layer of conductive adhesive covers all front edges of the sheet of graphite. At least a first region of the flexible backing that extends beyond the outer perimeter of the sheet of graphite has a self-adhesive front face that is configured to adhere to skin. The sheet of graphite has at least one elongated cutout disposed therein, with the at least one elongated cutout being positioned to increase flexibility of the electrode assembly when the electrode assembly is adhered to a subject's body. At least a second region of the flexible backing that is disposed behind the at least one elongated cutout has a self-adhesive front face that is configured to adhere to skin. And the at least one elongated cutout is at least 2 mm wide.

Another aspect of the invention is directed to a first apparatus for spreading heat within an electrode assembly. The first apparatus comprises a sheet of graphite that includes a main section, a first protruding section, and a second protruding section, with a gap disposed between the first and second protruding sections. The first and second protruding sections each extend into adjacent quadrants of a Cartesian coordinate system whose origin is positioned in the main section and whose Y axis is aligned with a centerline of the gap. The sheet of graphite has a front face. The main section includes a first region and a second region disposed on opposite sides of the Y axis, and the first and second protruding sections extend from the first and second regions, respectively. The first protruding section is at least half as long as a longest dimension of the first region in the Y direction, and the second protruding section is at least half as long as a longest dimension of the second region in the Y direction. The sheet of graphite has at least one slit disposed therein that extends inward from an outer edge of the sheet of graphite, and the at least one slit is positioned to increase flexibility of the first apparatus when the first apparatus is adhered to a subject's body.

In some embodiments of the first apparatus, the at least one slit comprises a central slit that extends inward from an outer edge of the main section that is opposite to the gap, the central slit is aligned with the Y axis, and the central slit extends at least one-tenth way through the main section.

In some embodiments of the first apparatus, the at least one slit comprises (i) a first slit positioned between the first region and the first protruding section (ii) a second slit positioned between the second region and the second protruding section, and (iii) a central slit that extends inward from an outer edge of the main section that is opposite to the gap and aligned with the Y axis. In these embodiments, the first and second slits extend inward from an outer edge of the sheet of graphite in directions that are perpendicular ±20° to the Y axis, and each of the first slit and the second slit and the central slit each extend at least one-tenth way through the main section.

Optionally, in the embodiments described in the previous paragraph, the sheet of graphite can have a first additional slit that extends in a proximal direction from a distal end of the first protruding section, and a second additional slit that extends in a proximal direction from a distal end of the second protruding section. Optionally, in the embodiments described in the previous paragraph, the first and second protruding sections are symmetric about the Y axis.

In some embodiments of the first apparatus, the first protruding section is at least as long as the longest dimension of the first region in the Y direction, and the second protruding section is at least as long as the longest dimension of the second region in the Y direction.

In some embodiments of the first apparatus, the main section, the first protruding section, and the second protruding section are collectively arranged in a configuration that is substantially U-shaped. In some embodiments of the first apparatus, the main section, the first protruding section, and the second protruding section are collectively arranged in a configuration that is substantially V-shaped or substantially rounded V-shaped.

In some embodiments of the first apparatus, the sheet of graphite has an area between 75 and 125cm² . In some embodiments of the first apparatus, the sheet of graphite is a sheet of pyrolytic graphite, graphitized polymer, or graphite foil made from compressed high purity exfoliated mineral graphite.

Some embodiments of the first apparatus further comprise a layer of conductive adhesive or conductive hydrogel disposed on the front face of the sheet of graphite. Some embodiments of the first apparatus further comprise a layer of flexible foam material shaped, dimensioned, and positioned to cover a front side of all edges of the sheet of graphite, and all of the slits in the sheet of graphite.

Another aspect of the invention is directed to a second apparatus for spreading heat within an electrode assembly. The second apparatus comprises a sheet of graphite that includes a main section, a first protruding section, and a second protruding section, with a gap disposed between the first and second protruding sections. The first and second protruding sections each extend into adjacent quadrants of a Cartesian coordinate system whose origin is positioned in the main section and whose Y axis is aligned with a centerline of the gap. The sheet of graphite has a front face. The main section includes a first region and a second region disposed on opposite sides of the Y axis, and the first and second protruding sections extend from the first and second regions, respectively. The first protruding section is at least half as long as a longest dimension of the first region in the Y direction, and the second protruding section is at least half as long as a longest dimension of the second region in the Y direction. The sheet of graphite has at least one slit or elongated cutout disposed therein that extends inward from an outer edge of the sheet of graphite, and the at least one slit or elongated cutout is positioned to increase flexibility of the apparatus when the apparatus is adhered to a subject's body.

In some embodiments of the second apparatus, the at least one slit or elongated cutout comprises a central slit or elongated cutout that extends inward from an outer edge of the main section that is opposite to the gap, the central slit or elongated cutout is aligned with the Y axis, and the central slit or elongated cutout extends at least one-tenth way through the main section.

In some embodiments of the second apparatus, the at least one slit or elongated cutout comprises (i) a first slit or elongated cutout positioned between the first region and the first protruding section (ii) a second slit or elongated cutout positioned between the second region and the second protruding section, and (iii) a central slit or elongated cutout that extends inward from an outer edge of the main section that is opposite to the gap and aligned with the Y axis. The first and second slits or elongated cutouts extend inward from an outer edge of the sheet of graphite in directions that are perpendicular ±20° to the Y axis, and each of the first slit or elongated cutout and the second slit or elongated cutout and the central slit or elongated cutout each extend at least one-tenth way through the main section.

In some embodiments of the second apparatus, the first protruding section is at least as long as the longest dimension of the first region in the Y direction, and the second protruding section is at least as long as the longest dimension of the second region in the Y direction.

In some embodiments of the second apparatus, the sheet of graphite has an area between 75 and 125cm^{2 .}

Some embodiments of the second apparatus further comprise a layer of conductive adhesive disposed on the front face of the sheet of graphite, and the layer of conductive adhesive covers all front edges of the sheet of graphite.

Some embodiments of the second apparatus further comprise a layer of conductive adhesive disposed on the front face of the sheet of graphite, and the layer of conductive adhesive extends at least 1 mm beyond all front edges of the sheet of graphite.

### BRIEF DESCRIPTION OF THE DRAWINGS

FIG. 1 depicts an exploded view of an electrode assembly that can be used to induce TTFields in the subject's body.

FIG. 2A depicts a rear view of the electrode assembly of FIG. 1, with the rearmost component removed.

FIG. 2B depicts a front view of the electrode assembly of FIG. 1, with the frontmost component removed.

FIG. 3 is a more detailed rear view of the electrode assembly of FIG. 1, with the rearmost component removed.

FIG. 4 depicts how the overall layout of the electrode assembly of FIG. 1 makes the electrode assembly bend more easily after it has been affixed to a subject's body.

FIG. 5 depicts one example of how to use the electrode assembly of FIG. 1 to apply TTFields to a subject's lungs.

FIG. 6 depicts another example of how to use the electrode assembly of FIG. 1 to apply TTFields to a subject's lungs.

FIG. 7 depicts an example of how to use the electrode assembly of FIG. 1 to treat a subject with pancreatic cancer using TTFields.

FIGS. 8 and 9 are, respectively, perspective and plan views of a set of components that can be used in place of certain other components within the electrode assembly of FIG. 1.

Various embodiments are described in detail below with reference to the accompanying drawings, wherein like reference numerals represent like elements.

### DESCRIPTION OF THE PREFERRED EMBODIMENTS

As explained above, some TTFields electrode assemblies use a sheet of graphite to spread out the heat and electrical current, with a layer of conductive adhesive or conductive hydrogel positioned between the sheet of graphite and the subject's body to hold (or help hold) the electrode assembly to the subject's skin. However, because graphite sheets do not stretch (and act more like a sheet of paper than a flexible fabric bandage), normal movements of the subject's body in certain anatomical locations can cause the electrode assemblies to peel away from the subject body. And this will impair or interrupt the TTFields treatment.

One anatomic location where electrode assemblies have a relatively high tendency to peel away from the subject's body is the center of the sternum (due to breathing activity and the stretching of skin in that area when the person moves their arms). Another anatomic location where electrode assemblies have a relatively high tendency to peel away is the side of the abdomen (due to the stretching of skin in that area when the person rotates their torso or leans to either side).

The embodiments described below have features that can advantageously reduce the electrode assemblies'tendency to peel away from the subject's skin in these and other anatomical locations.

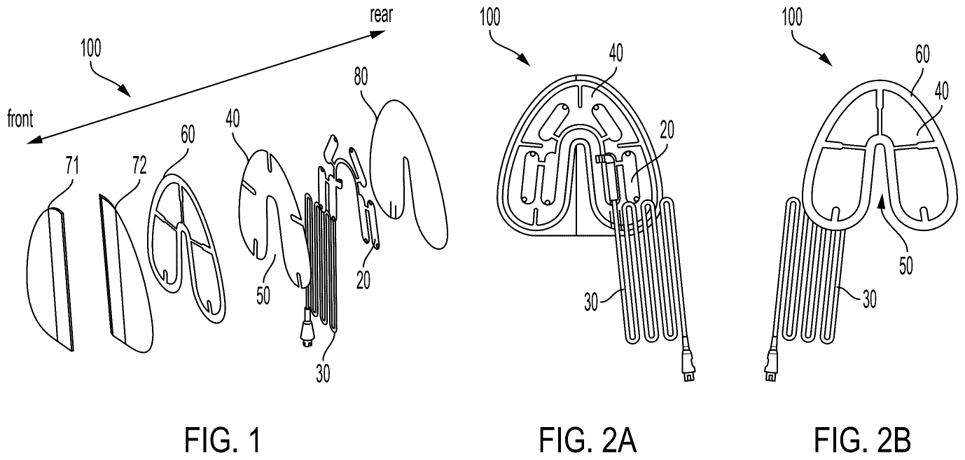

FIG. 1 depicts an exploded view of an electrode assembly 100 that, when positioned on a subject's body, can be used to induce TTFields in the subject's body. The electrode assembly 100 includes (progressing from the rear to the front) a flexible backing 80, a flexible PCB 20 (i.e., a flexible printed circuit board, which is commonly referred to as a “flex circuit”) with a cable 30 that terminates on the flexible PCB 20, a sheet of graphite 40 (having a gap 50), an optional layer of foam material 60, and a cover 71, 72 (e.g., a release liner). During use, the cover 71, 72 is removed.

FIG. 2A depicts a rear view of the electrode assembly 100 with the flexible backing 80 removed so that the components in front of the flexible backing 80 can be seen. And FIG. 2B depicts a front view of the electrode assembly 100 with the cover 71, 72 removed so that the components positioned behind the cover 71, 72 can be seen.

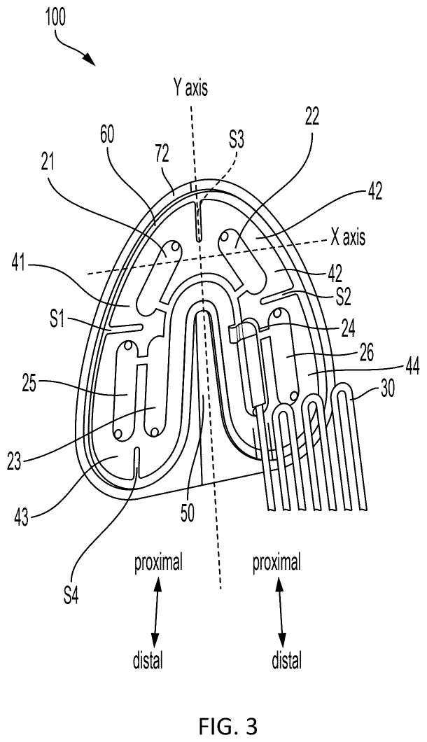

FIG. 3 is a more detailed view of the electrode assembly 100 with the flexible backing 80 removed. The sheet of graphite 40 spreads out both heat and current in the plane of the page in FIGS. 2A and 2B. The sheet of graphite 40 includes a main section (which includes a first region 41 and a second region 42), a first protruding section 43, and a second protruding section 44, and there is a gap 50 disposed between the first and second protruding sections 43, 44.

Examples of suitable materials for the sheet of graphite 40 include, but are not limited to, synthetic graphite, pyrolytic graphite (including, but not limited to, Pyrolytic Graphite Sheet (PGS), available from Panasonic Industry, Kadoma, Osaka, Japan), graphitized polymer film (e.g., graphitized polyimide film, including, but not limited to, that supplied by Kaneka Corp., Moka, Tochigi, Japan), or graphite foil made from compressed high purity exfoliated mineral graphite (including, but not limited to, that supplied by MinGraph® 2010A Flexible Graphite, available from Mineral Seal Corp., Tucson, Arizona, USA). The sheet of graphite 40 spreads out both heat and current in the plane of the page in FIGS. 2A and 2B. In some embodiments, a layer of a different conductive anisotropic material may be used in place of the sheet of graphite 40.

The first and second protruding sections 43, 44 each extend into adjacent quadrants of a Cartesian coordinate system (denoted by the dashed lines in FIG. 3) whose origin is positioned in the main section and whose Y axis is aligned with a centerline of the gap 50. The first region 41 and the second region 42 of the main section 41, 42 are disposed on opposite sides of the Y axis, and the first and second protruding sections 43, 44 extend from the first and second regions 41, 42, respectively. In the example depicted in FIG. 3, the first and second protruding sections 43, 44 are longer in the Y direction than a longest dimension of the first and second regions 41, 42 in the Y direction, respectively. But in alternative embodiments, the first and second protruding section 43, 44 can be at least one-quarter as long (or at least half as long, or at least as long) as a longest dimension of the first and second regions 41, 42 in the Y direction, respectively. The sheet of graphite 40 has a front face and a rear face.

In some embodiments, the sheet of graphite 40 has an area between 50 and 200cm² . But in other embodiments, the sheet of graphite 40 can have an area of 75-100 cm², 75-125 cm², 75-150 cm², 75-200 cm², 75-250 cm², 75-300 cm², 50-100 cm², 50-125 cm², 50-150 cm ², 50-250 cm², 50-300 cm², 30-100 cm², 30-125 cm², 30-150 cm², 30-200 cm², or 30-300 cm², or even an area that is not within any of these ranges.

In the embodiment depicted in FIG. 3, the main section 41, 42, the first protruding section 43, and the second protruding section 44 of the sheet of graphite 40 can be collectively arranged in a configuration that is substantially rounded V-shaped. But this configuration is not the only suitable configuration. Examples of other suitable configurations for the sheet of graphite 40 include but are not limited to configurations in which the main section 41, 42, the first protruding section 43, and the second protruding section 44 of the sheet of graphite 40 are collectively arranged in a configuration that is substantially U-shaped or substantially V-shaped.

The PCB 20 is positioned behind the rear face of the sheet of graphite 40, with the front face of the PCB facing toward the sheet of graphite. The PCB has a first metal pad 21 that is disposed on the front face of the PCB and positioned behind the first region 41, a second metal pad 22 that is disposed on the front face of the PCB and positioned behind the second region 42, a third metal pad 23 that is disposed on the front face of the PCB and positioned behind the first protruding section 43, and a fourth metal pad 24 that is disposed on the front face of the PCB and positioned behind the second protruding section 44. In the embodiment depicted in FIG. 3, fifth and sixth metal pads 25, 26 are disposed on the front face of the PCB 20 and positioned behind the first and second protruding sections 43, 44, respectively. But these fifth and sixth metal pads 25, 26 are optional and can be omitted.

The first and second metal pads 21, 22 of the PCB are affixed to the first and second regions 41, 42 of the sheet of graphite 40, respectively, by first and second regions of a conductive adhesive or conductive hydrogel. And the third and fourth metal pads 23, 24 of the PCB are affixed to the first and second protruding sections 43, 44 of the sheet of graphite 40, respectively, by third and fourth regions of a conductive adhesive or conductive hydrogel. When the fifth and sixth metal pads 25, 26 are included, they are affixed to the first and second protruding sections 43, 44 of the sheet of graphite 40, respectively, by corresponding regions of the conductive adhesive or conductive hydrogel.

Examples of suitable materials for the conductive adhesive or conductive hydrogel include, but are not limited to, the OMNI-WAVE™ adhesive compositions manufactured and sold by FLEXCON® (Spencer, MA, USA), such as the developmental product FLX068983—FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M, Saint Paul, MN, USA) may also be used.

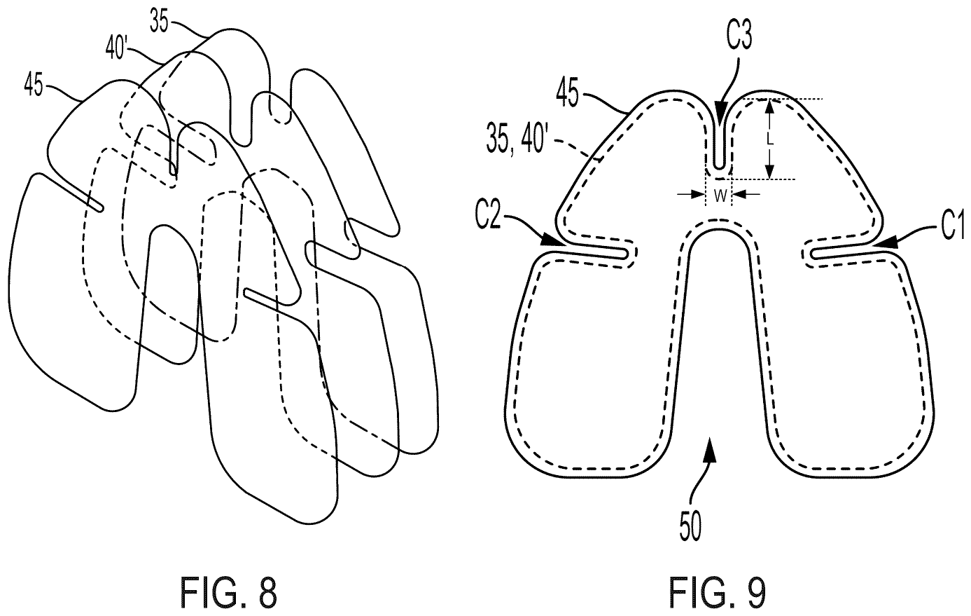

Note that the sheet of graphite 40 can be affixed to the PCB 20 by positioning a single layer of conductive adhesive between the sheet of graphite 40 and the PCB 20 (see, for example, conductive adhesive 35, as shown and described with respect to FIGS. 8 and 9). In this case the metal pads 21-24 of the PCB would be affixed to the first, second, third, and fourth regions 41-44 of the sheet of graphite 40, respectively, by first, second, third, and fourth regions of the single layer of conductive adhesive. But in alternative embodiments, additional layers of conductive material can be positioned between the sheet of graphite 40 and the PCB 20. For example, two layers made from different types of conductive adhesives (i.e., a front layer and a rear layer) can be positioned between the sheet of graphite 40 and the PCB 20. In this situation, the front layer affixes the regions 41-44 of the sheet of graphite 40 to the corresponding metal pads 21-24 of the PCB 20, and the rear layer also affixes the regions 41-44 of the sheet of graphite 40 to the corresponding metal pads 21-24 of the PCB 20. Alternatively, a layer of conductive adhesive (front layer or rear layer) and a layer of conductive hydrogel (front layer or rear layer) may be used.

A front layer of conductive adhesive or conductive hydrogel (not shown) is preferably disposed on the front face of the sheet of graphite, and this layer will help the electrode assembly 100 adhere to the subject's skin (see, e.g., the conductive adhesive 45 described below in connection with FIGS. 8 and 9). The same materials described above in connection with the conductive adhesive or conductive hydrogel that sits between the sheet of graphite 40 and the PCB 20 can be used on the front face of the sheet of graphite.

The electrode assembly 100 depicted in FIG. 3 has five slits S1-S5 disposed in the sheet of graphite 40, and these slits are positioned to increase flexibility of the electrode assembly 100 when the electrode assembly is adhered to a subject's body.

More specifically, the sheet of graphite 40 in the embodiment depicted in FIG. 3 has (i) a first slit S1 positioned between the first region 41 and the first protruding section 43 and (ii) a second slit S2 positioned between the second region 42 and the second protruding section 44. Each of these slits extends inward from an outer edge of the sheet of graphite 40 in a direction that is perpendicular ±10° to the Y axis, and these slits S1-S2 each extend at least one-tenth way through the main section 41, 42. In alternative embodiments, the direction of these slits S1, S2 can be perpendicular ±20° to the Y axis.

The sheet of graphite 40 in the embodiment depicted in FIG. 3 also has a central slit S3 that extends inward from an outer edge of the main section 41, 42 that is opposite to the gap 50, and this central slit S3 is aligned with the Y axis. In the illustrated embodiment, the central slit S3 is precisely aligned with the Y axis, and it extends between one-third and halfway through the main section 41, 42. But in alternative embodiments, the central slit can be less precisely aligned with the Y axis (e.g., ±15°), and extends at least one-tenth way through the main section.

The sheet of graphite 40 in the embodiment depicted in FIG. 3 also has a first additional slit S4 that extends in a proximal direction from a distal end of the first protruding section 43, and a second additional slit S5 that extends in a proximal direction from a distal end of the second protruding section 44.

Note that while the electrode assembly 100 depicted in FIG. 3 has five slits S1-S5 disposed in the sheet of graphite 40, the configuration of slits depicted in that figure is not the only configuration that can be used. To the contrary, a wide variety of different configurations for the slits can be used, including but not limited to using only a single slit (e.g., the slit S3 described above), using only two slits (e.g., the slits S1 and S2 described above), using only three slits (e.g., the slits S1-S3 described above), or using slits S1-S3 described above combined with additional slits that are not shown. In some embodiments (e.g., as described below in connection with FIGS. 8 and 9), elongated cutouts that are significantly wider than the relatively narrow slits S1-S5 depicted in FIG. 3 are used.

Note also that while the first and second protruding sections 43, 44 of the electrode assembly 100 depicted in FIG. 3 are symmetric about the Y axis, those protruding sections 43, 44 need not be symmetric about the Y axis.

Optionally, and as best seen in FIGS. 1 and 2B, the electrode assembly 100 also has a layer of flexible foam material 60 shaped, dimensioned, and positioned to cover a front side of all edges of the sheet of graphite 40, and all of the slits S1-S5. The purpose of this layer of flexible foam material 60 is to cover all the edges of the sheet of graphite 40 because those edges can be sharp, and covering those edges can prevent the subject from getting small cuts. This foam material can be similar to the foam material that is used in foam self-adhesive bandages. Note that when the layer of flexible foam material 60 is omitted, it is preferable to employ another approach (e.g., the approach described below in connection with FIGS. 8 and 9) for preventing small cuts.

The electrode assembly 100 depicted in FIGS. 1-3 also has a flexible backing 80 positioned behind the PCB 20, and this flexible backing is configured to support the PCB and the sheet of graphite. This flexible backing 80 may have a self-adhesive front face and can be made of a variety of materials including but not limited to flexible fabric materials, foam materials, and plastic materials (e.g., similar to corresponding varieties of Band-Aid® brand adhesive bandages). Note, however, that in alternative embodiments, the flexible backing 80 can be omitted.

The electrode assembly 100 depicted in FIGS. 1-3 also has a front cover 71, 72 positioned in front of the layer of conductive adhesive (or hydrogel) that sits in front of the sheet of graphite 40. This cover 71, 72 (release liner) performs a similar function to the coated-paper slips that cover Band-Aid® brand adhesive bandages, and it prevents dust and dirt from settling on the front layer of adhesive before the electrode assembly 100 is applied to the subject's skin. Note, however, that in alternative embodiments, the cover 71, 72 can be omitted.

The PCB 20 has a plurality of metal traces configured to form electrically conductive paths between the first metal pad 21, the second metal pad 22, the third metal pad 23, and the fourth metal pad 24. And the electrode assembly 100 also has a cable 30 that drives the first, second, third, and fourth metal pads 21-24. These conductive wires and traces form paths for signals from an AC signal generator (not shown) to arrive at the metal pads 21-24 via the cable 30.

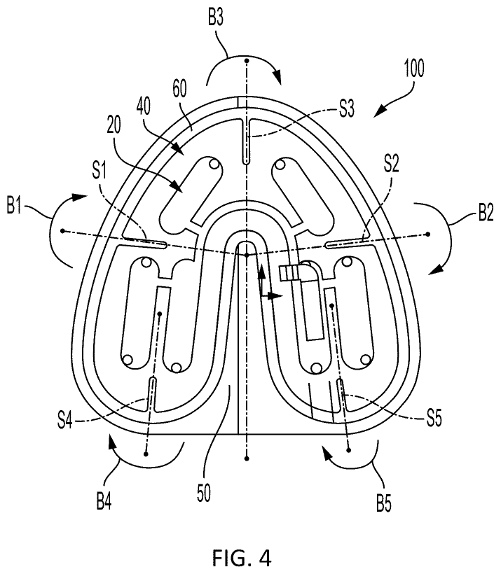

FIG. 4 depicts how the overall layout of the electrode assembly 100, and in particular the positions of the gap 50 and the slits S1-S5 contribute to making the electrode assembly bend more easily after it has been affixed to a subject's body. More specifically, the position of the gap 50 and the central slit S3 make the electrode assembly 100 bend more easily about the bending axis indicated by the arrow B3; and the positions of the slits S1, S2, S4, and S5 make the electrode assembly 100 bend more easily about the bending axes indicated by the arrows B1, B2, B4, and B5, respectively. Bendability of the electrode assembly 100 can be further enhanced by ensuring that none of the bending axes pass through any of the large metal pads of the PCB (as is the case for the electrode assembly depicted in FIG. 4).

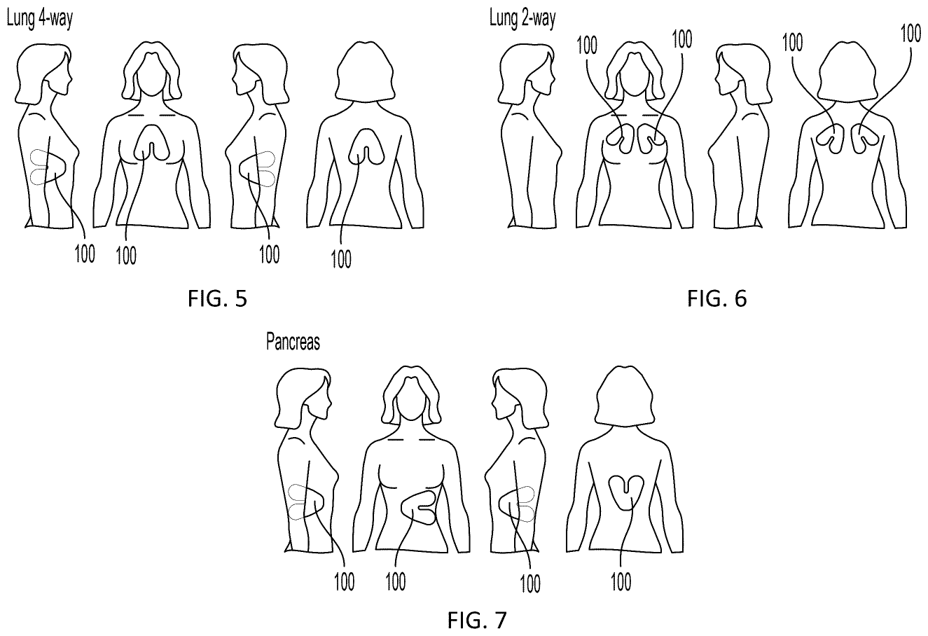

FIG. 5 depicts one example of how to use the electrode assembly 100 described above in connection with FIGS. 1-4 to apply TTFields to a subject's lungs. In this example, four electrode assemblies 100 are positioned in front, in back, to the right, and to the left of the subject's lungs. When an AC voltage is applied between the front and back electrode assemblies 100, an AC current is coupled through those electrode assemblies and into the subject's body, which induces TTFields with a first direction within the subject's lungs. And when an AC voltage is applied between the left and right electrode assemblies 100, an AC current is coupled through those electrode assemblies and into the subject's body, which induces TTFields with a different direction within the subject's lungs.

Notably, when the front electrode assembly 100 is positioned and oriented as indicated in FIG. 5 (i.e., centered over the subject's sternum with the gap 50 facing down), the improved bendability of the electrode assembly will prevent the front electrode assembly 100 from peeling away from the subject's body when the subject breathes and/or moves their arms. Similarly, when the rear electrode assembly 100 is positioned and oriented as indicated in FIG. 5, the improved bendability of the electrode assembly will prevent the rear electrode assembly 100 from peeling away from the subject's body when the subject moves their arms and/or shoulders.

FIG. 6 depicts an alternative layout for positioning four electrode assemblies 100 in a subject that has lung cancer, and FIG. 7 depicts a suitable layout for positioning four electrode assembles 100 in a subject with pancreatic cancer. In each case, each of the electrode assemblies 100 is positioned and oriented as indicated in the corresponding figure, and the improved bendability of the electrode assemblies will prevent them from peeling away from the subject's body when the subject breathes and or/moves.

Importantly, while the electrode assembly 100 described above can be made and sold to the end customer in its entirety, each electrode assembly can also be made and sold as two or more discrete subassemblies that can be assembled (for example, adhered to each other) and used together as a single electrode assembly by the end customer just prior to use. One example of this would be to divide the electrode assembly 100 depicted in FIG. 1 into two subassemblies as follows: (1) a front subassembly that includes the sheet of graphite 40, the layer of foam material 60, and the cover 71/72; and (2) a rear subassembly that includes the flexible backing 80, the PCB 20, and the cable 30. Optionally, when the electrode assembly 100 is divided into subassemblies along those lines, additional coverings that resemble the cover 71/72 may be positioned in front of the PCB 20 and behind the sheet of graphite 40 to preserve the condition of the adhesive layers on the front of the flexible backing 80 and on the rear of the sheet of graphite 40.

The advantage of dividing the electrode assembly 100 along these lines is that the front subassembly (which is less expensive to manufacture) can be disposable, and the rear subassembly (which is more expensive) can be reused more than once by pressing a new front subassembly onto the same rear subassembly before each use. An adhesive layer positioned on the rear of the sheet of graphite 40 and/or the front of the rear subassembly will make the front and rear subassemblies stick to each other. In this situation, the rear subassembly will interface with the signal generator so that the signal generator can apply a voltage to the pads of the PCB 20. And the front subassembly will provide a path for the electrical current to enter the subject's body, spread heat and current out over the entire surface of the sheet of graphite 40, and prevent ions (e.g., calcium ions) from entering or leaving the subject's body.

The rear view of the front subassembly will resemble the view depicted in FIG. 3, except that the PCB 20 and the cable 30 will not be included. The sheet of graphite 40 will therefore include a main section (which includes a first region 41 and a second region 42), a first protruding section 43, and a second protruding section 44, and there is a gap 50 disposed between the first and second protruding sections 43, 44. The sheet of graphite in this embodiment can be made from the same materials described above in connection with FIG. 3.

As described above in connection with FIG. 3, the first and second protruding sections 43, 44 each extend into adjacent quadrants of a Cartesian coordinate system (denoted by the dashed lines in FIG. 3) whose origin is positioned in the main section and whose Y axis is aligned with a centerline of the gap 50. The first region 41 and the second region 42 of the main section 41, 42 are disposed on opposite sides of the Y axis, and the first and second protruding sections 43, 44 extend from the first and second regions 41, 42, respectively. In the example depicted in FIG. 3, the first and second protruding sections 43, 44 are longer in the Y direction than a longest dimension of the first and second regions 41, 42 in the Y direction, respectively. But in alternative embodiments, the first and second protruding section 43, 44 can be at least half as long (or the same length) as a longest dimension of the first and second regions 41, 42 in the Y direction, respectively. The sheet of graphite 40 has a front face, and the area of the sheet of graphite in this embodiment can be as described above in connection with FIG. 3.

The main section 41, 42, the first protruding section 43, and the second protruding section 44 of the sheet of graphite 40 can be collectively arranged into any of the configurations described above in connection with FIG. 3 (e.g., substantially U-shaped, substantially V-shaped, or substantially rounded V-shaped).

The sheet of graphite 40 in the front subassembly has at least one slit disposed therein that extends inward from an outer edge of the sheet of graphite, and the at least one slit is positioned to increase flexibility of the front subassembly when the front subassembly is adhered to a subject's body. The at least one slit could be all five of the slits S1-S5 depicted in FIG. 3 and described above. It could also be only a single slit (e.g., the slit S3 described above), only two slits (e.g., the slits S1 and S2 described above), only three slits (e.g., the slits S1-S3 described above), or slits S1-S3 described above combined with additional slits that are not shown in FIG. 3.

Other details for the front subassembly are similar to those described above in connection with the components 40-72 of the FIG. 3 embodiment described above.

As noted above, when the layer of flexible foam material 60 is omitted, it is preferable to employ another approach for preventing small cuts. FIGS. 8 and 9 are, respectively, perspective and plan views of the sheet of graphite 40′ and the adjacent layers of adhesive that are used in one such approach. In this approach, the components depicted in FIGS. 8 and 9 take the place of the sheet of graphite 40 and the layer of flexible foam material 60 depicted in FIGS. 1 and 2B. All the other components described above in connection with FIGS. 1-4 retain their original positions and functions.

The sheet of graphite 40′ in this embodiment is very similar to the sheet of graphite 40 described above in connection with FIGS. 1-3, except that in the place of the relatively narrow slits S1-S3 described above, this embodiment has elongated cutouts C1-C3 that are significantly wider. These elongated cutouts are positioned to increase flexibility of the electrode assembly when the electrode assembly is adhered to a subject's body. As used herein, the phrase “elongated cutout” refers to a cutout with a longitudinal length that is at least 50% larger than its width. See, e.g., FIG. 9, which uses the labels L and W for the longitudinal length and width, respectively.

Note that while FIGS. 8 and 9 depict three elongated cutouts C1-C3 disposed at particular positions, the quantity, position, and orientation of the elongated cutouts can vary e.g., as described above for the slits S1-S5 in the FIGS. 1-4 embodiments. The elongated cutouts C1-C3 in these embodiments improve the bendability of the electrode assembly and prevent the electrode assembly from peeling away from the subject body for reasons similar to those described above in connection with FIGS. 1-7.

One layer of conductive adhesive 35 is disposed on the rear face of the sheet of graphite 40′ and those two layers are positioned in direct contact with each other. This layer of conductive adhesive 35 affixes the metal pads 21-24 of the PCB to respective regions 41-44 of the sheet of graphite 40′, as described above in connection with FIGS. 1-3.

Returning to FIGS. 8 and 9, another layer of conductive adhesive 45 is disposed on the front face of the sheet of graphite 40′ and those two layers are positioned in direct contact with each other. As best seen in FIG. 9, the layer of conductive adhesive 45 is slightly larger than the sheet of graphite 40′, and those two layers are aligned so that the layer of conductive adhesive 45 covers all the front edges of the sheet of graphite 40′. One preferred material for the layer of conductive adhesive 45 is ARcare® 8006 electrically conductive adhesive composition manufactured and sold by Adhesives Research, Inc. (Glen Rock, PA, USA). However, any of the other conductive adhesives discussed above in connection with FIGS. 1-3 could also be used in this embodiment.

Notably, when the layer of conductive adhesive 45 covers all the front edges of the sheet of graphite 40′ (e.g., by extending at least 1 mm or at least 2 mm beyond all front edges of the sheet of graphite), those edges will no longer be able to cut into the subject's skin. This embodiment therefore effectively and advantageously prevents the small cuts described above without including a dedicated component for that purpose. More specifically, the layer of conductive adhesive 45 in this embodiment performs two functions: (i) holding the sheet of graphite 40′ to the subject body, and (ii) preventing the front edges of the sheet of graphite 40′ from cutting the subject's skin. In contrast, those two functions are performed by respective different structures in the FIGS. 1-4 embodiment described above.

As explained above, some embodiments of the electrode assembly have a flexible backing 80 positioned behind the PCB 20. This flexible backing is configured to support the PCB and the sheet of graphite 40/40′. When a portion of the flexible backing 80 extends beyond an outer perimeter of the sheet of graphite 40/40′, and when that portion has a self-adhesive front face that is configured to adhere to skin, the flexible backing 80 will hold (or help hold) the electrode assembly against the subject's body. And when the front face of the flexible backing 80 has a stronger adhesive than the layer of conductive adhesive 45, the contribution of the flexible backing 80 towards holding the electrode assembly against the subject's body will be even more significant.

In some preferred embodiments, the flexible backing 80 is fabricated so that regions of the flexible backing that are disposed behind the elongated cutouts C1-C3 in the sheet of graphite 40′ have a self-adhesive front face that is configured to adhere to skin. In these embodiments, the elongated cutouts C1-C3 have widths W that are wide enough (e.g., ≥2 mm, ≥3 mm, ≥4 mm, ≥5 mm, ≥6 mm, ≥8 mm, ≥10 mm) so that the self-adhesive front face of the flexible backing 80 can contact the subject's skin through the elongated cutouts. This configuration increases the overall area of the front face of the flexible backing 80 that makes contact with the subject's skin, which enhances the flexible backing's ability to hold the electrode assembly against the subject's body. Optionally, the inner end of each of the elongated cutouts C1-C3 can have a rounded, slightly enlarged region similar to a buttonhole. This configuration can further increase the overall area of the front face of the flexible backing 80 that makes contact with the subject's skin.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An electrode assembly comprising:
a sheet of graphite that includes a main section, a first protruding section, and a second protruding section, with a gap disposed between the first and second protruding sections,
wherein the first and second protruding sections each extend into adjacent quadrants of a Cartesian coordinate system whose origin is positioned in the main section and whose Y axis is aligned with a centerline of the gap,
wherein the sheet of graphite has a front face and a rear face,
wherein the main section includes a first region and a second region disposed on opposite sides of the Y axis, and wherein the first and second protruding sections extend from the first and second regions, respectively,
wherein the first protruding section is at least one-quarter as long as a longest dimension of the first region in the Y direction, and
wherein the second protruding section is at least one-quarter as long as a longest dimension of the second region in the Y direction; and
a flexible PCB positioned behind the rear face of the sheet of graphite, wherein the PCB has a front face facing toward the sheet of graphite,
a first metal pad that is disposed on the front face of the PCB and positioned behind the first region,
a second metal pad that is disposed on the front face of the PCB and positioned behind the second region,
a third metal pad that is disposed on the front face of the PCB and positioned behind the first protruding section, and
a fourth metal pad that is disposed on the front face of the PCB and positioned behind the second protruding section,
wherein the first and second metal pads of the PCB are affixed to the first and second regions of the sheet of graphite, respectively, by first and second regions of a conductive adhesive or conductive hydrogel, and
wherein the third and fourth metal pads of the PCB are affixed to the first and second protruding sections of the sheet of graphite, respectively, by third and fourth regions of a conductive adhesive or conductive hydrogel.

2. The electrode assembly of claim 1, wherein the PCB has a plurality of metal traces configured to form electrically conductive paths between the first metal pad, the second metal pad, the third metal pad, and the fourth metal pad, and
wherein the electrode assembly further comprises a conductive wire that is electrically connected to at least one of the first, second, third, and fourth metal pads.

3. The electrode assembly of claim 2, further comprising a layer of conductive adhesive disposed on the front face of the sheet of graphite.

4. The electrode assembly of claim 3, further comprising a flexible backing positioned behind the PCB, wherein the flexible backing is configured to support the PCB and the sheet of graphite.

5. The electrode assembly of claim 1, wherein the sheet of graphite has at least one slit or elongated cutout disposed therein, and the at least one slit or elongated cutout is positioned to increase flexibility of the electrode assembly when the electrode assembly is adhered to a subject's body.

6. The electrode assembly of claim 1, wherein the sheet of graphite has a central slit or elongated cutout that extends inward from an outer edge of the main section that is opposite to the gap, wherein the central slit or elongated cutout is aligned with the Y axis.

7. The electrode assembly of claim 1, wherein the sheet of graphite has (i) a first slit or elongated cutout positioned between the first region and the first protruding section and (ii) a second slit or elongated cutout positioned between the second region and the second protruding section,
wherein the first and second slits or elongated cutouts extend inward from an outer edge of the sheet of graphite in directions that are perpendicular ±20° to the Y axis, and
wherein the first and second slits or elongated cutouts each extend at least one-tenth way through the main section.

8. The electrode assembly of claim 7, wherein the sheet of graphite has a central slit or elongated cutout that extends inward from an outer edge of the main section that is opposite to the gap,
wherein the central slit or elongated cutout is aligned with the Y axis, and
wherein the central slit or elongated cutout extends at least one-tenth way through the main section.

9. The electrode assembly of claim 1, wherein the first protruding section is at least as long as the longest dimension of the first region in the Y direction, and
wherein the second protruding section is at least as long as the longest dimension of the second region in the Y direction.

10. The electrode assembly of claim 1, wherein the sheet of graphite has an area between 50 and 200cm².

11. The electrode assembly of claim 1, further comprising:
a flexible backing positioned behind the PCB, wherein the flexible backing is configured to support the PCB and the sheet of graphite, and wherein the flexible backing extends beyond an outer perimeter of the sheet of graphite; and
a layer of conductive adhesive disposed on the front face of the sheet of graphite, wherein the layer of conductive adhesive covers all front edges of the sheet of graphite,
wherein at least a first region of the flexible backing that extends beyond the outer perimeter of the sheet of graphite has a self-adhesive front face that is configured to adhere to skin.

12. The electrode assembly of claim 11, wherein the sheet of graphite has at least one elongated cutout disposed therein, with the at least one elongated cutout being positioned to increase flexibility of the electrode assembly when the electrode assembly is adhered to a subject's body,
wherein at least a second region of the flexible backing that is disposed behind the at least one elongated cutout has a self-adhesive front face that is configured to adhere to skin, and
wherein the at least one elongated cutout is wide enough to permit the second region of the flexible backing to contact the subject's skin.

13. The electrode assembly of claim 11, wherein the sheet of graphite has at least one elongated cutout disposed therein, with the at least one elongated cutout being positioned to increase flexibility of the electrode assembly when the electrode assembly is adhered to a subject's body,
wherein at least a second region of the flexible backing that is disposed behind the at least one elongated cutout has a self-adhesive front face that is configured to adhere to skin, and
wherein the at least one elongated cutout is at least 2 mm wide.

14. An apparatus for spreading heat within an electrode assembly, the apparatus comprising:
a sheet of graphite that includes a main section, a first protruding section, and a second protruding section, with a gap disposed between the first and second protruding sections,
wherein the first and second protruding sections each extend into adjacent quadrants of a Cartesian coordinate system whose origin is positioned in the main section and whose Y axis is aligned with a centerline of the gap,
wherein the sheet of graphite has a front face,
wherein the main section includes a first region and a second region disposed on opposite sides of the Y axis, and wherein the first and second protruding sections extend from the first and second regions, respectively,
wherein the first protruding section is at least half as long as a longest dimension of the first region in the Y direction, and
wherein the second protruding section is at least half as long as a longest dimension of the second region in the Y direction,
wherein the sheet of graphite has at least one slit or elongated cutout disposed therein that extends inward from an outer edge of the sheet of graphite, and wherein the at least one slit or elongated cutout is positioned to increase flexibility of the apparatus when the apparatus is adhered to a subject's body.

15. The apparatus of claim 14, wherein the at least one slit or elongated cutout comprises a central slit or elongated cutout that extends inward from an outer edge of the main section that is opposite to the gap,
wherein the central slit or elongated cutout is aligned with the Y axis, and
wherein the central slit or elongated cutout extends at least one-tenth way through the main section.

16. The apparatus of claim 14, wherein the at least one slit or elongated cutout comprises (i) a first slit or elongated cutout positioned between the first region and the first protruding section (ii) a second slit or elongated cutout positioned between the second region and the second protruding section, and (iii) a central slit or elongated cutout that extends inward from an outer edge of the main section that is opposite to the gap and aligned with the Y axis,
wherein the first and second slits or elongated cutouts extend inward from an outer edge of the sheet of graphite in directions that are perpendicular ±20° to the Y axis,
wherein each of the first slit or elongated cutout and the second slit or elongated cutout and the central slit or elongated cutout each extend at least one-tenth way through the main section.

17. The apparatus of claim 14, wherein the first protruding section is at least as long as the longest dimension of the first region in the Y direction, and
wherein the second protruding section is at least as long as the longest dimension of the second region in the Y direction.

18. The apparatus of claim 14, wherein the sheet of graphite has an area between 75 and 125cm².

19. The apparatus of claim 14, further comprising a layer of conductive adhesive disposed on the front face of the sheet of graphite, wherein the layer of conductive adhesive covers all front edges of the sheet of graphite.

20. The apparatus of claim 14, further comprising a layer of conductive adhesive disposed on the front face of the sheet of graphite, wherein the layer of conductive adhesive extends at least 1 mm beyond all front edges of the sheet of graphite.
